(19) United States
(12) Patent Application Publication
(10) Pub. No.: US 2025/0325807 A1
(43) Pub. Date: Oct. 23, 2025

(54) DEVICES AND METHODS FOR SENSING ANALYTES AND DELIVERING THERAPEUTIC AGENTS

(71) Applicant: Dexcom, Inc., San Diego, CA (US)

(72) Inventor: Joshua Windmiller, San Diego, CA (US)

(73) Assignee: Dexcom, Inc., San Diego, CA (US)

(21) Appl. No.: 19/253,153

(22) Filed: Jun. 27, 2025

Related U.S. Application Data
(63) Continuation of application No. PCT/US23/78073, filed on Oct. 27, 2023.
(60) Provisional application No. 63/436,476, filed on Dec. 30, 2022.

(51) Int. Cl. A61N 1/32; A61N 1/04; A61N 1/30; A61N 1/36

(52) U.S. Cl. CPC A61N 1/325; A61N 1/0412; A61N 1/0448; A61N 1/306; A61N 1/327; A61N 1/36031; A61N 1/36034

(57) ABSTRACT

Some examples herein provide a wearable device for sensing a concentration of an analyte and delivering a therapeutic agent. A sensor is configured to extend fully through stratum corneum, epidermis, and dermis and partially into subcutaneous tissue, and includes a distal end configured to be located within the subcutaneous tissue. A reservoir is configured to contact the stratum corneum and includes a polymer complexed with the drug. Control electronics coupled to the sensor's proximal end of the sensor include first and second electrodes, and are configured to receive a signal from the sensor's distal end corresponding to the concentration of the analyte within the subcutaneous tissue. Control electronics determine, using the signal, electrical stimulus to be applied to a first electrode and a second electrode, and apply that electrical stimulus to deliver the therapeutic agent across the stratum corneum.

### CROSS-REFERENCE TO RELATED APPLICATIONS

This application is a continuation of International Patent Application No. PCT/US23/78073 filed Oct. 27, 2023, which claims the benefit of U.S. Provisional Application No. 63/436,476 filed Dec. 30, 2022, the entirety of which is incorporated herein by reference.

### FIELD

This application generally relates to devices and methods for sensing one or more analytes and delivering one or more therapeutic agents.

### BACKGROUND

CGM wearables are adhered to the skin by means of a medical-grade adhesive. Oftentimes, CGM is used in patients with intensive insulin therapy, many of whom wear patch pumps or infusion sets containing said medical-grade adhesives. In these situations, the CGM is used to inform the delivery of insulin to counteract elevated glucose levels. However, this requires users to adorn a minimum of two devices on the body.

Several methods for delivering a therapeutic agent to a host's skin have been established, including microneedles, iontophoresis, electroporation, laser ablation, radiofrequency ablation, and ultrasound ablation. Such methods may overcome the barrier function of the stratum corneum, to deliver a specific quantity of the therapeutic agent to the host's skin. However, it would be useful to have improved control over the amount of therapeutic agent that is delivered.

### SUMMARY

Wearable devices for sensing one or more analytes and delivering one or more therapeutic agents, and methods of using the same, are provided herein. The wearable device includes a sensor configured to extend fully through a stratum corneum, epidermis, and dermis of a host and partially into subcutaneous tissue of the host. The sensor includes a proximal end and a distal end. The distal end is configured to be positioned within the subcutaneous tissue. The wearable device may include at least one reservoir configured to contact the stratum corneum and may include a polymer complexed with the therapeutic agent. The wearable device includes control electronics coupled to the proximal end of the sensor and includes a first electrode and a second electrode. The control electronics are configured to, via the proximal end of the sensor, receive a signal from the distal end of the sensor corresponding to a concentration of one or more analytes within the subcutaneous tissue. The control electronics are configured to determine, using the signal, an electrical stimulus to be applied to the first electrode and second electrode. The control electronics are configured to apply the electrical stimulus to the first and second electrodes to deliver the therapeutic agent across the stratum corneum. In this manner, closed-loop control for sensing analyte concentration(s) and delivering therapeutic agent(s) is provided in a single wearable device.

The device further includes a housing within which the proximal end of the sensor and the control electronics are disposed.

In some examples, wherein responsive to the electrical stimulus, an amount of the therapeutic agent is transported out of the polymer. In some examples, responsive to the electrical stimulus, the amount of the therapeutic agent is transported through the stratum corneum and into the epidermis. In some examples, responsive to the electrical stimulus, the amount of the therapeutic agent is transported through the stratum corneum and epidermis and into the dermis.

In some examples, applying the electrical stimulus to the first and second electrodes delivers the therapeutic agent through the stratum corneum and into the epidermis via iontophoresis. In some examples, applying the electrical stimulus to the first and second electrodes delivers the therapeutic agent into the epidermis via electroporation. In some examples, applying the electrical stimulus to the first and second electrodes delivers the therapeutic agent into the epidermis via magnetohydrodynamics.

In some examples, the therapeutic agent is charged. In some examples, the therapeutic agent is positively charged. In some examples, the therapeutic agent is negatively charged.

In some examples, the therapeutic agent has a neutral charge and is carried by a charged carrier. In some examples, the charged carrier is positively charged. In some examples, the charged carrier is negatively charged.

In some examples, a first reservoir of the at least one reservoir is adjacent to the first electrode. In some examples, a second reservoir of the at least one reservoir is located at a spaced distance from the first reservoir. In some examples, the second reservoir is adjacent to the second electrode. In some examples, both the first and second reservoirs include the polymer complexed with the therapeutic agent. In some examples, the electrical stimulus alternates as a function of time to alternately transport the therapeutic agent out of the first reservoir and the second reservoir.

In some examples, the first reservoir includes the polymer complexed with the therapeutic agent, and the second reservoir includes a second polymer. In some examples, the electrical stimulus alternates as a function of time to alternately transport the therapeutic agent out of the first reservoir and transport a counterion into the second reservoir.

In some examples, the electrical stimulus is substantially constant to transport the therapeutic agent out of the first reservoir and transport a counterion into the second reservoir.

In some examples, the electrical stimulus substantially does not interfere with the signal corresponding to the concentration of the analyte within the subcutaneous tissue.

In some examples, the control electronics receives the signal at a time during which the electrical stimulus is not being applied.

In some examples, the sensor is located between the first and second electrodes.

In some examples, the second electrode is located between the sensor and the first electrode.

In some examples, the sensor is located in an aperture within the first electrode.

In some examples, the first electrode is located in an aperture within the second electrode.

In some examples, the proximal end of the sensor is less than about 1 cm away from at least one of the first and second electrodes.

In some examples, the control electronics is configured to determine the electrical stimulus based on a duration of time for which the at least one reservoir has been coupled to the stratum corneum. In some examples, the control electronics is configured to increase a duration of the electrical stimulus as the duration of time for which the at least one reservoir has been coupled to the stratum corneum increases. In some examples, the control electronics is configured to increase a magnitude of the electrical stimulus as the duration of time for which the at least one reservoir has been coupled to the stratum corneum increases.

In some examples, the control electronics is configured to determine the electrical stimulus responsive to the signal differing from a predetermined value by more a predetermined amount.

In some examples, the analyte includes a metabolite of the therapeutic agent. In some examples, the analyte includes a metabolite of insulin, levodopa, metformin, glucagon, GLP-1 antagonist, SGLT-2 inhibitor, vancomycin, gentamycin, epinephrine, or naloxone.

In some examples, the therapeutic agent includes insulin, levodopa, metformin, glucagon, GLP-1 antagonist, SGLT-2 inhibitor, vancomycin, gentamycin, epinephrine, or naloxone.

In some examples, the device further includes adhesive configured to adhere the sensor and the control electronics to the epidermis. In some examples, the at least one reservoir is located within the adhesive.

Some examples herein provide a method for delivering a therapeutic agent. The method may include, by control electronics of a wearable device adhered to a stratum corneum of a host, receiving a signal from a distal end of a sensor of the wearable device via a proximal end of the sensor which is coupled to the control electronics. In one example, the distal end of the sensor is located within subcutaneous tissue of the host, and the signal may correspond to a concentration of an analyte within the subcutaneous tissue. The method may include, by the control electronics, using the signal to determine an electrical stimulus to be applied between first and second electrodes of the wearable device. The method may include, by the control electronics, applying the electrical stimulus to the first and second electrodes to transport an amount of the therapeutic agent out of at least one reservoir of the wearable device, through the stratum corneum and epidermis, and into the dermis for uptake of the therapeutic agent by capillaries in the dermis.

### BRIEF DESCRIPTION OF DRAWINGS

FIGS. 1A-1E schematically illustrate example configurations of, and operations performed by, a wearable device, consistent with implementations of the present disclosure.

FIG. 2A schematically illustrates a bottom view of the wearable device example configuration of FIG. 1E, consistent with implementations of the present disclosure.

FIG. 2B schematically illustrates an example of an electric field between the electrodes of the wearable device of FIG. 1E, consistent with implementations of the present disclosure.

FIGS. 3-6 schematically illustrate alternative example configurations of, and operations performed by, a wearable device.

FIGS. 7A-7B schematically illustrate additional alternative example configurations of, and operations performed by, a wearable device for delivering a therapeutic agent.

FIGS. 8A-8B, and 9A-9B schematically illustrate additional alternative example configurations of, and operations performed by, a wearable device for delivering a therapeutic agent.

FIGS. 9A-9B schematically illustrate additional alternative example configurations of, and operations performed by, a wearable device for delivering a therapeutic agent.

FIG. 10 illustrates a flow of operations in an example method for delivering a therapeutic agent using a wearable device.

### DETAILED DESCRIPTION

Wearable devices for sensing one or more analytes and delivering one or more therapeutic agents, and methods of using the same, are provided herein. For example, the wearable device includes a sensor for measuring an analyte concentration, a reservoir storing a therapeutic agent, and control electronics for determining the analyte concentration and administering the therapeutic agent from the reservoir and into the skin of a host. It is appreciated that a single sensor may include a plurality of working electrodes for measuring a plurality of analyte concentrations, or a plurality of sensors each measuring an analyte concentration, as part of a single wearable device. The control electronics measure the analyte concentration in order to determine the amount of the therapeutic agent to be administered, for example by determining the time and/or magnitude of an electrical stimulus to apply to the reservoir which releases that amount of the therapeutic agent and/or the rate at which the therapeutic agent is released. In this manner, the wearable device can more effectively titrate the dosing for maximal therapeutic efficacy in a manner that would be too burdensome for a user to manage on their own initiative. Further, by integrating both analyte measurement and therapeutic agent delivery into a single wearable device, accurate amounts and/or rates of the therapeutic agent are deliverable directly into the skin on a rapid, as-needed basis without the need for the host's involvement or intervention. Indeed, the host may not necessarily even be aware of when the therapeutic agent is being delivered. In comparison, some previously known methods of administering therapeutic agents involve the host measuring an analyte concentration, using that information to separately determine a dose of the therapeutic agent to administer, and then separately administering the dose. Such previously known methods constitute a significant burden on the host, and the amount and/or rate of therapeutic agent the host administers may be inaccurate due to miscalculations, or due to delays between when the measurement is made and when the therapeutic agent ultimately is administered. Also in comparison to previously known systems, two devices spaced a sufficient distance apart on the host must be worn to separately sense an analyte and dose a therapeutic agent without interference. For example, insulin pumps are spaced apart from continuous glucose monitors so that insulin preservatives, which are electroactive species, do not interfere with the glucose concentration signal. Such previously known systems pose a significant burden on the host, and require the purchase and maintenance of two separate wearable devices. Accordingly, it will be appreciated that the present wearable devices and methods, in one example, continuously monitor the concentration of any suitable analyte in a physiologic fluid of a user (e.g., blood, interstitial fluid) from anywhere (e.g., at home, work, while traveling, or other locations) and automatically administer an appropriate amount and/or rate of therapeutic agent with the same device and without the need for the host's intervention (or, optionally, knowledge), which provides the host with an improved outcome and/or reduced burden of management of a disease or health condition.

First, some example terms used in the present application will be explained. Then, example wearable devices for delivering a drug to a host, and methods of using such devices, will be provided.

### Terms

In order to facilitate an understanding of the disclosed examples, a number of terms are defined below.

The term “about” as used herein is a broad term, and is to be given its ordinary and customary meaning to a person of ordinary skill in the art (and is not to be limited to a special or customized meaning), and refers without limitation to allowing for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt. % to about 5 wt. % of the composition is the material, or about 0 wt. % to about 1 wt. %, or about 5 wt. % or less, or less than or equal to about 4.5 wt. %, 4, 3.5, 3, 2.5, 2, 1.5, 1, 0.9, 0.8, 0.7, 0.6, 0.5, 0.4, 0.3, 0.2, 0.1, 0.01, or about 0.001 wt. % or less, or about 0 wt. %.

The terms “adhere” and “attach” as used herein are broad terms, and are to be given their ordinary and customary meaning to a person of ordinary skill in the art (and are not to be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, metabolites, and/or reaction products. In some examples, the analyte measured by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bilirubin, biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine; creatine kinase; creatine kinase MM isoenzyme; creatinine; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycerol; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, β); lysozyme; mefloquine; netilmicin; oxygen; phenobarbitone; phenytoin; phytanic/pristanic acid; potassium, sodium, and/or other blood electrolytes; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uric acid; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, or endogenous, for example, a metabolic product, a hormone, an antigen, an antibody, and the like. Alternately, the analyte can be introduced into the body, or exogenous, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, RITALIN®, CYLERT®, PRELUDIN®, DIDREX®, PRESTATE®, VORANIL®, SANDREX®, PLEGINE®); depressants (barbiturates, methaqualone, tranquilizers such as VALIUM®, LIBRIUM®, MILTOWN®, SERAX®, EQUANIL®, TRANXENE®); hallucinogens (phencyclidine, lysergic acid, mescaline, peyote, psilocybin); narcotics (heroin, codeine, morphine, opium, meperidine, PERCOCET®, PERCODAN®, TUSSIONEX®, fentanyl, DARVON®, TALWIN, LOMOTIL®); designer drugs (analogs of fentanyl, meperidine, amphetamines, methamphetamines, and phencyclidine, for example, Ecstasy); anabolic steroids; and nicotine. The metabolic products of the aforementioned drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and histamine.

The phrases “analyte-measuring device,” “analyte-monitoring device,” “analyte-sensing device,” “continuous analyte sensing device,” “continuous analyte sensor device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing and/or detecting element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The phrase “barrier cell layer” as used herein is a broad phrase, and is to be given its ordinary and customary meaning to a person of ordinary skill in the art (and is not to be limited to a special or customized meaning), and refers without limitation to a part of a foreign body response that forms a cohesive monolayer of cells (for example, macrophages and foreign body giant cells) that substantially block the transport of molecules and other substances to the implantable device.

The term “baseline” and “background” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a sensor that is irrespective of the concentration of the measured analyte or is otherwise the signal produced with no analyte present.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, continuous analyte sensing device, continuous analyte sensor device, and/or multi-analyte sensor device responsible for the detection of, or transduction of a signal associated with, a particular analyte or combination of analytes. In examples, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In examples, such biosensors and/or sensors are capable of providing specific quantitative, semi-quantitative, qualitative, semi qualitative analytical signals using a biological recognition element combined with a detecting and/or transducing element.

Various examples of sensor architectures can be found in pending U.S. Application No. 63/321,538, titled, “CONTINUOUS ANALYTE SENSOR SYSTEMS,” filed Mar. 17, 2022., incorporated by reference in its entirety herein, as well as U.S. Pat. No. 8,133,178 to Brauker, et al., which is incorporated herein by reference in its entirety, as well as U.S. Pat. No. 8,828,201, Simpson, et al.; U.S. Pat. No. 9,131,885 Simpson, et al.; U.S. Pat. No. 9,237,864, Simpson, et al.; and U.S. Pat. No. 9,763,608, Simpson, et al., each of which is incorporated by reference in its entirety herein. Examples of methods of forming the sensors (sensor electrode layouts and membrane) and sensor systems discussed herein can be found in currently pending U.S. Patent Pub. 2019/0307371 to Boock, et al., incorporated by reference in its entirety herein.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The term “coaxial” as used herein is to be construed broadly to include sensor architectures having elements aligned along a shared axis around a core that can be configured to have a circular, elliptical, triangular, polygonal, or other cross-sections, such elements can include electrodes, insulating layers, or other elements that can be positioned circumferentially around the core layer, such as a core electrode or core polymer wire.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer of sensor systems as discussed herein.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains of system systems as discussed herein.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The phrase “continuous analyte sensing” as used herein is a broad phrase, and is to be given its ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every 5 seconds or less to about 10 minutes or more. In further examples, monitoring of analyte concentration is performed from about every 10, 15, 20, 25, 30, 35, 40, 45, 50, 55, or 60 seconds to about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In some examples, monitoring of analyte concentration is performed about every 15 minutes, or about every 30 minutes, or about every 60 minutes; additionally, or alternatively, in some examples, monitoring of analyte concentration is performed about every 1.5 hours, about every 2 hours, about every 4 hours, about every 6 hours, or about every 8 hours.

The term “complexed” as used herein is a broad term, and is to be given its ordinary and customary meaning to a person of ordinary skill in the art (and is not to be limited to a special or customized meaning), and refers without limitation to two or more system elements or components that are configured to be at least one of mechanically, covalently, ionically, or otherwise chemically bound. In some examples, a therapeutic agent is chemically bound in a polymer. In some examples, a therapeutic agent is mechanically bound in a polymer.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. Similarly, the phrases “operably connected”, “operably linked”, and “operably coupled” as used herein, refer to one or more components linked to another component(s) in a manner that facilitates transmission of at least one signal between the components. In some examples, components are part of the same structure and/or integral with one another (i.e. “directly coupled”). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein, refer to two or more system elements or components that are configured to be or have been electrically, mechanically, thermally, operably, chemically, or otherwise attached and detached without damaging any of the coupled elements or components. The phrase “permanently coupled” as used herein, refer to two or more system elements or components that are configured to be or have been electrically, mechanically, thermally, operably, chemically, or otherwise attached but cannot be uncoupled without damaging at least one of the coupled elements or components.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of the membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In various examples, a byproduct of a reaction of an analyte being detected includes at least one measurable species. The at least one measurable species can react with an electrochemically active surface, such as a working electrode.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term “host” as used herein is a broad term, and is to be given its ordinary and customary meaning to a person of ordinary skill in the art (and is not to be limited to a special or customized meaning), and refers without limitation to mammals, for example humans.

The terms “indwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The phrase “insertable volume” as used herein is a broad phrase, and is to be given its ordinary and customary meaning to a person of ordinary skill in the art (and is not to be limited to a special or customized meaning), and refers without limitation to a volume ahead of and alongside a path of insertion of an insertable portion of an analyte sensor, as described herein, as well as an incision made in the skin to insert the insertable portion of the analyte sensor. The insertable volume also includes up to 5 mm radially or perpendicular to the volume ahead of and alongside the path of insertion.

The terms “interferants,” and “interfering species” as used herein are broad terms, and are to be given their ordinary and customary meaning to a person of ordinary skill in the art (and are not to be limited to a special or customized meaning), and refer without limitation to effects and/or species, including ions, electroactive substances, endogenous circulating species, exogenous circulating species, pharmacologic agents, and/or electromagnetic waves (such as from a magnetic resonance imaging (MRI) system or medical device) that interfere with the measurement of an analyte of interest in a sensor to produce a signal that does not accurately represent the analyte measurement.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, that is constructed of materials of a few microns thickness or more, which is permeable to at least the analyte the concentration of which is to be measured.

The term “micro,” as used herein is a broad term, and is to be given its ordinary and customary meaning to a person of ordinary skill in the art (and is not to be limited to a special or customized meaning), and refers without limitation to a small object or scale of approximately 10⁻⁶ to 10⁻³ m that is not visible without magnification. The term “micro” is in contrast to the term “macro” that refers to a large object that may be visible without magnification. Similarly, the term “nano” refers to a small object or scale of approximately 10⁻⁹ to 10⁻⁶ m.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The term “planar” as used herein is to be interpreted broadly to describe sensor architecture having a substrate including a first side and a second side, and a plurality of elements arranged on one or more sides of the substrate, the elements may or may not be electrically or otherwise coupled, where the elements can include conductive or insulating layers or elements configured to operate as a circuit.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The phrase “sensing membrane” as used herein is a broad phrase, and is to be given its ordinary and customary meaning to a person of ordinary skill in the art (and is not to be limited to a special or customized meaning), and refers without limitation to a permeable or semi-permeable membrane that can comprise one or more domains, layers, or layers within domains and that is constructed of materials having a thickness of a few microns or more, and that are permeable to reactants and/or co-reactants employed in determining the analyte of interest.

The phrases “sensing portion,” “sensing membrane,” “sensing region,” “sensing domain,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In examples, the sensing portion, sensing membrane, and/or sensing mechanism generally comprise an electrode configured to provide signals during electrochemically reactions with one or more membranes covering electrochemically reactive surface. In examples, such sensing portions, sensing membranes, and/or sensing mechanisms are capable of providing specific quantitative, semi-quantitative, qualitative, semi qualitative analytical signals using a biological recognition element combined with a detecting and/or transducing element.

In one example, the sensing region determines the selectivity among one or more analytes, so that only the analyte which has to be measured leads to (transduces) a detectable signal. In one example, the selection is based on any chemical or physical recognition of the analyte by the sensing region, where the chemical composition of the analyte is unchanged, or in which the sensing region causes or catalyzes a reaction of the analyte that changes the chemical composition of the analyte.

The sensing region transduces the recognition of analytes into a semi-quantitative or quantitative signal. Thus, “transducing” or “transduction” and their grammatical equivalents as are used herein encompasses electrochemical technologies and methods. Electrochemical properties include current and/or voltage, capacitance, resistance, impedance, charge, and potential.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, an amperometric sensor has a sensitivity (or slope) of from about 1 to about 100 picoAmps of current for every 1 mg/dL of analyte.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a coaxial or wire-type sensor, wherein the diameter is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a planar or substantially planar substrate, wherein the thickness of the implantable portion is less than about 1 mm, see, for example, U.S. Pat. No. 6,175,752 to Say et al. and U.S. Pat. No. 5,779,665 to Mastrototaro et. al., both of which are incorporated herein by reference in their entirety. Examples of methods of forming sensors (sensor electrode layouts and membrane) and sensor systems which can used to prepare the present sensors can be found in US Patent Pub 2019/0307371 to Boock et al., incorporated by reference in its entirety herein.

The term and phrase “zwitterion” and “zwitterionic compound” as used herein are each a broad term and phrase, and are to be given their ordinary and customary meaning to a person of ordinary skill in the art (and is not to be limited to a special or customized meaning), and refer without limitation to compounds in which a neutral molecule of the compound has a unit positive and unit negative electrical charge at different locations within the molecule. Such compounds are a type of dipolar compound, and are also sometimes referred to as “inner salts.”

### Wearable Devices for Delivering a Therapeutic Agent, and Methods of Using the Same

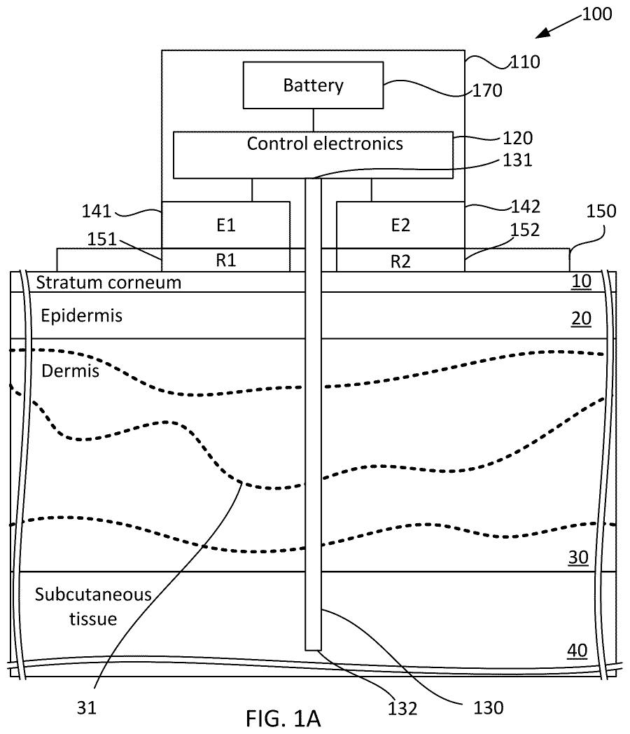

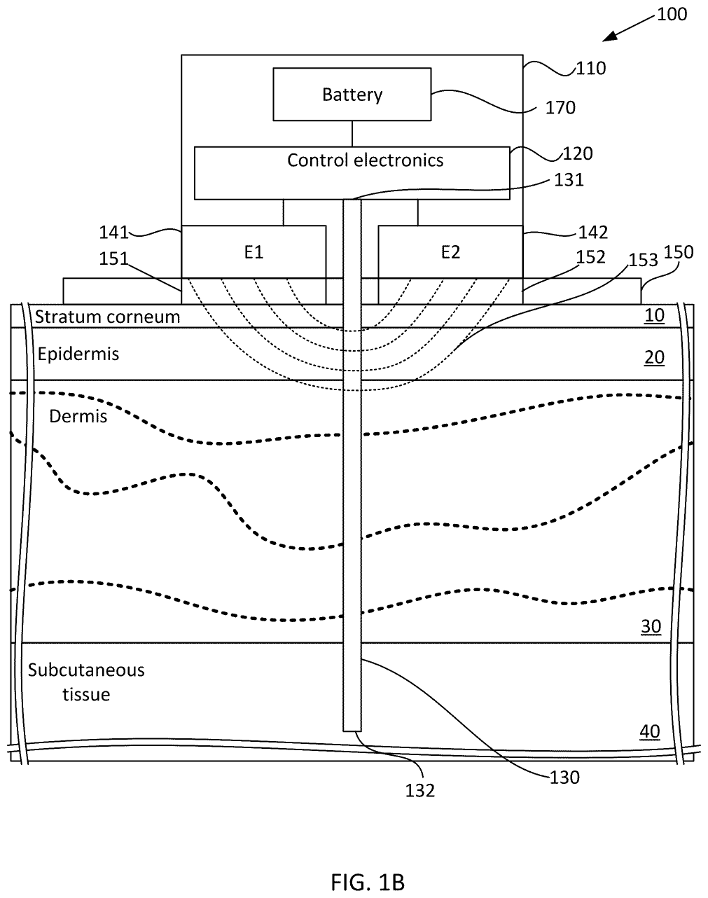

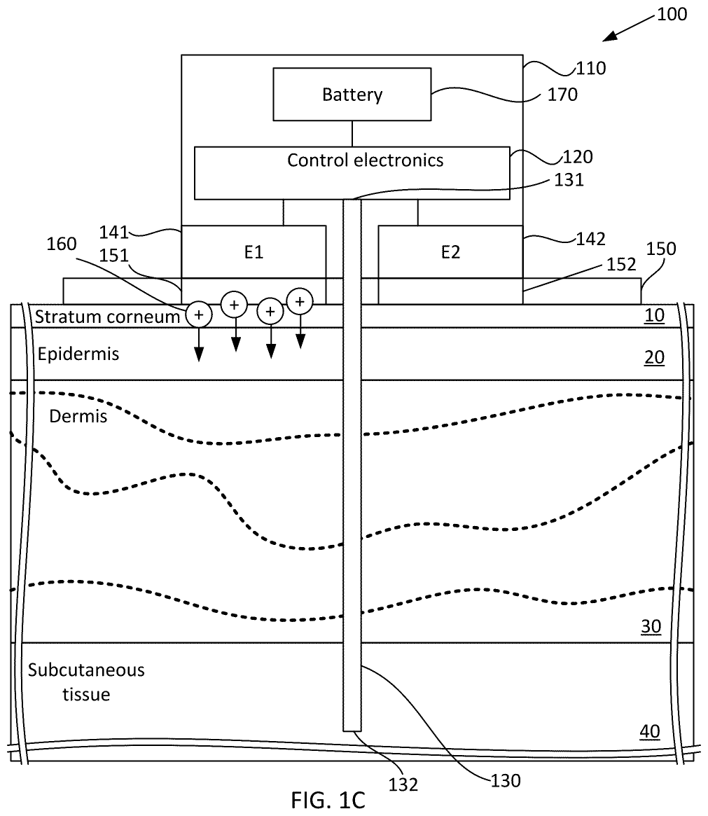

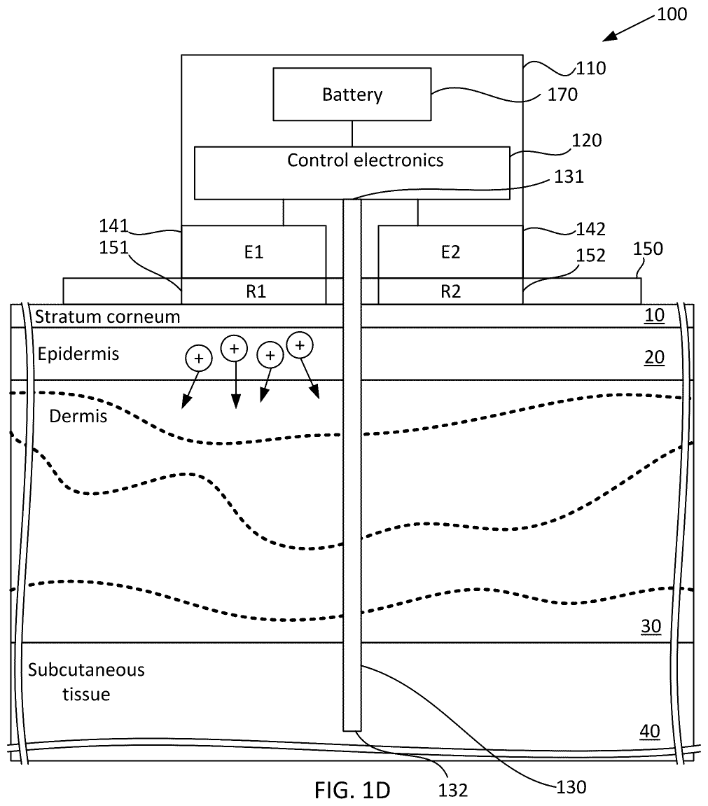

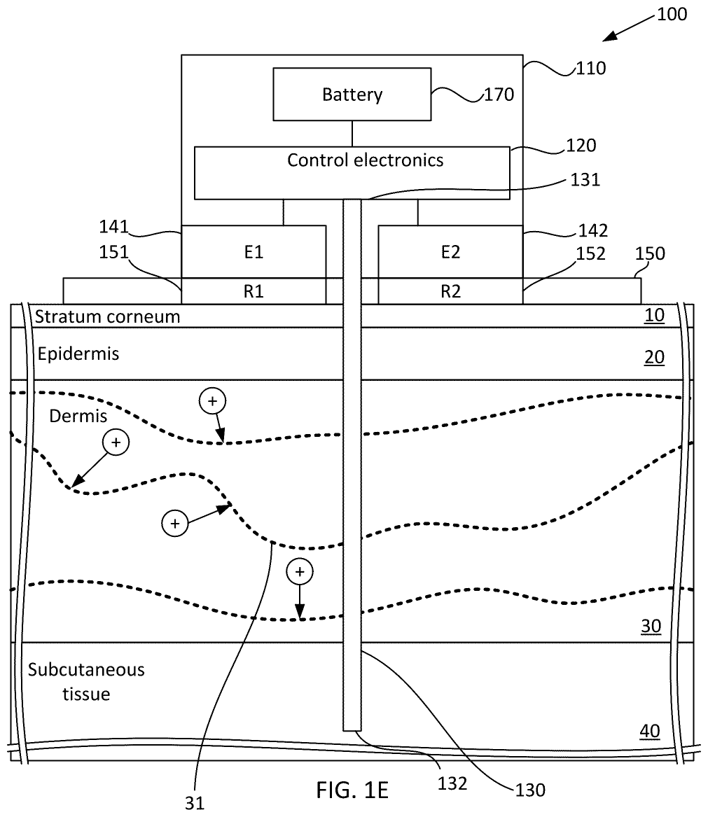

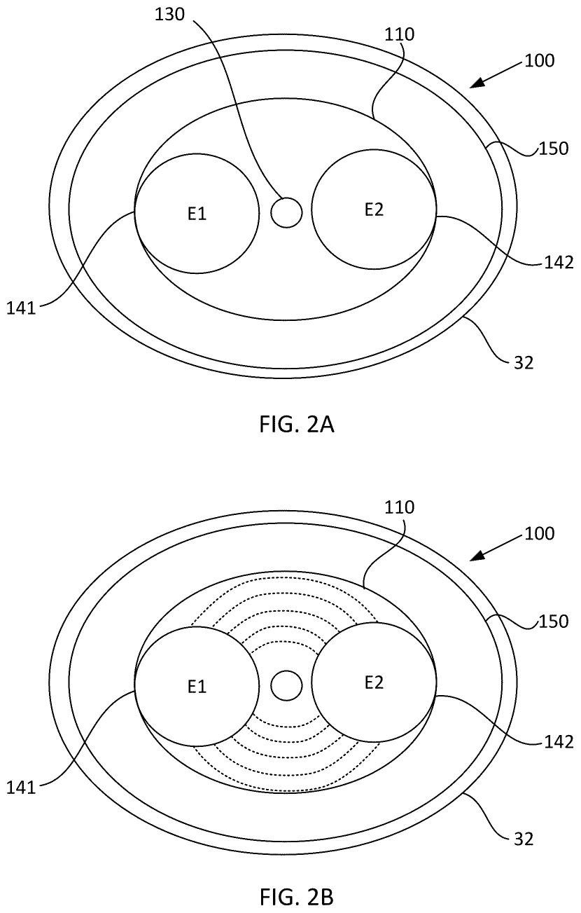

Nonlimiting examples of devices and methods for measuring a physiological signal and/or concentration of a target analyte in vivo now will be described with reference to FIGS. 1A-1E, 2A-2B, 3-6, 7A-7B, 8A-8B, 9A-9B, and 10. FIGS. 1A-1E and 2A-2B schematically illustrate an example configuration of, and operations performed by, a wearable device for sensing an analyte and delivering a therapeutic agent. Turning first to FIG. 1A, wearable device 100 includes housing 110, control electronics 120, sensor 130, and at least one reservoir (e.g., the first reservoir 151 and the second reservoir 152). In one example, wearable device 100 also includes battery 170 coupled to control electronics 120 and configured to provide power thereto. In one example, wearable device 100 is configured to be coupled or adhered to stratum corneum 10 (which is a part of epidermis 20) using, for example, a suitable biocompatible adhesive pad 32 as illustrated in FIG. 1A or by other coupling means such as an elastic strap or adjustable wrist ban. For example, footplate 150 or elastic strap or adjustable wrist ban is configured to secure wearable device 100 including the sensor 130 and the control electronics 120 to the epidermis 20. In one example, footplate 150 comprises one or both of first reservoir 151 and the second reservoir 152.

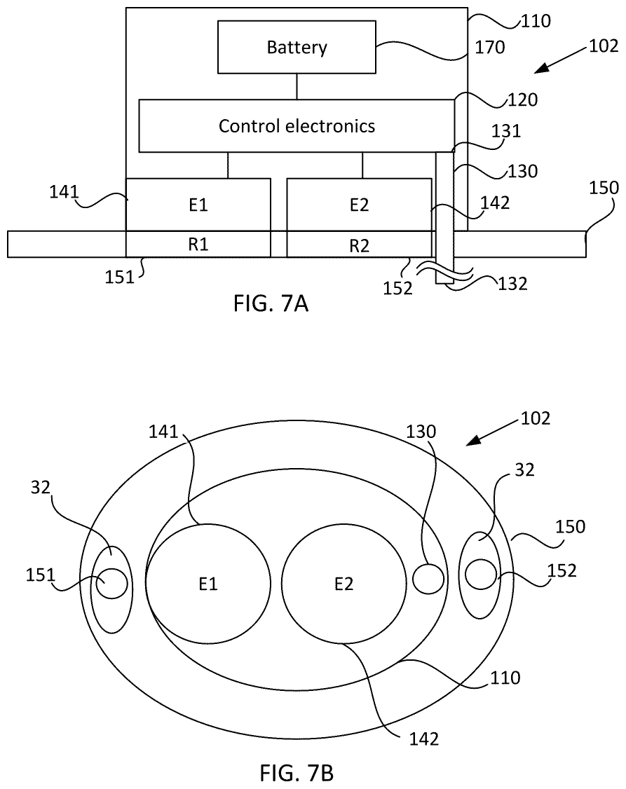

FIG. 2A illustrates a partial plan view of wearable device 100. In the nonlimiting example illustrated in FIGS. 1A and 2A, sensor 130 is located between a first electrode 141 and a second electrode 142. In the illustrated in FIG. 1A, a first reservoir 151 is adjacent to the first electrode 141, a second reservoir 152 is located at a spaced distance from the first reservoir 151, and the second reservoir 152 is adjacent to the second electrode 142. However, in other examples, such as described further below with reference to FIGS. 7A-7B, 8A-8B, 9A-9B, sensor 130, the first electrode 141 and the second electrode 142, and the first reservoir 151 and the second reservoir 152 is arranged in other configurations and spatial relationships relative to one another. In one example, as depicted in FIG. 2A, the housing 110 is generally circular and adhesive pad 32 is of the same or slightly larger circular diameter of the housing.

Sensor 130 is configured to extend fully through stratum corneum 10, epidermis 20, and dermis 30 of a host and partially into subcutaneous tissue 40 of the host. For example, sensor 130 includes a proximal end 131 and a distal end 132. Distal end 132 is configured to be located within subcutaneous tissue 40 of the host, and proximal end 131 is coupled to control electronics 120 within housing 110. Distal end 132 includes a first working electrode 133, and optionally a second working electrode 134, and optionally additional working electrodes (not shown). The at least one reservoir optionally includes a polymer complexed with a therapeutic agent. In the nonlimiting example illustrated in FIG. 1A, at least one of the first reservoir 151 and the second reservoir 152 (which also can be denoted R1 and R2, respectively) includes a polymer complexed with the therapeutic agent. In certain other examples such as described further below, both of the first reservoir 151 and the second reservoir 152 optionally stores the therapeutic agent. In one example, control electronics 120 are coupled to proximal end 131 of sensor 130 and include a first electrode 141 and a second electrode 142 (which also can be denoted E1 and E2, respectively). Control electronics 120 are configured to, via proximal end 131 of sensor 130, receive a signal from the distal end 132 of the sensor 130 corresponding to a concentration of an analyte within the subcutaneous tissue 40.

In one example, control electronics 120 also are configured to determine, using the signal, an electrical stimulus to be applied to the first electrode 141 and the second electrode 142; and to apply the electrical stimulus to the first electrode 141 and the second electrode 142. As such, control electronics 120 are configured and used to automatically control delivery of a therapeutic agent out of the at least one reservoir (e.g., out of first reservoir 151 and/or second reservoir 152) into the dermis, responsive to the concentration of an analyte which the distal end 132 of the sensor 130 measured within the subcutaneous tissue 40. For example, at the particular time illustrated in FIG. 1B and FIG. 2B, control electronics 120 applies a voltage and a current between the first electrode 141 and the second electrode 142, which generates electrical field lines 153 that penetrate stratum corneum 10 and fully or partially penetrate the balance of epidermis 20 as well. Responsive to the electrical stimulus, an amount of the therapeutic agent is transported out of the polymer of the first reservoir 151 and/or the second reservoir 152. For example, the electrical fields generated through application of the electrical stimulus causes release of the therapeutic agent from the first reservoir 151 and/or the second reservoir 152. At a particular time illustrated in FIG. 1C, the electrical stimulus causes movement of therapeutic agent 160 out of reservoir 151 and across stratum corneum 10. In one example, the therapeutic agent is positively charged, e.g., as illustrated in FIG. 1C, or the therapeutic agent has no charge (i.e., it is neutral) and be carried by a positively charged carrier. Alternatively, the therapeutic agent is negatively charged, or the therapeutic agent has no charge (i.e., it is neutral) and is carried by a negatively charged carrier. In such examples, the carrier includes a charged species with which the therapeutic agent is complexed, or includes a charged encapsulant in which the therapeutic agent is disposed therein. The electrical stimulus causes the transport of the therapeutic agent 160 into epidermis 20 and dermis 30; alternatively, the therapeutic agent 160 is transported into epidermis 20 and dermis 30 via diffusion or circulation. For example, at the particular time illustrated in FIG. 1D, therapeutic agent 160 is transported into epidermis 20 and into dermis 30. At the particular time illustrated in FIG. 1E, therapeutic agent 160 is transported into dermis 30 where capillaries 31 uptake the therapeutic agent for systemic distribution. By dosing and sensing in different layers of skin and tissue, i.e. dosing the therapeutic agent 160 into epidermis 20 and dermis 30, and sensing the analyte in the subcutaneous tissue, potential interference due to sensing the therapeutic agent 160 and/or is constituents is avoided.

Any suitable analyte can be measured, and any suitable therapeutic agent can be delivered, using wearable device 100. Non-limiting examples of analytes are provided elsewhere herein. In some examples, the analyte which is measured by wearable device 100 is selected from the group consisting of glucose, lactate, ketone bodies (such as acetoacetate, acetone, or beta-hydroxybutyrate), ions (such as sodium, potassium, calcium, magnesium, or chloride), or hormones (such as insulin or cortisol). In some examples, the analyte measured using wearable device 100 includes a metabolite of the therapeutic agent, for example a metabolite of insulin, levodopa, metformin, glucagon, GLP-1 antagonist, SGLT-2 inhibitor, vancomycin, gentamycin, epinephrine, or naloxone. Nonlimiting examples of therapeutic agents that are deliverable using wearable device 100 include insulin, levodopa, metformin, glucagon, GLP-1 antagonist, SGLT-2 inhibitor, vancomycin, gentamycin, epinephrine, or naloxone.

It will be appreciated that control electronics 120 are configured to control the delivery of the therapeutic agent 160 via different methods. In some examples, control electronics 120 apply the electrical stimulus to the first electrode 141 and the second electrode 142 to deliver the therapeutic agent 160 into the dermis 30 via electroporation. In other examples, control electronics 120 apply the electrical stimulus to the first electrode 141 and the second electrode 142 to deliver the therapeutic agent 160 into the dermis 30 via iontophoresis. For further details regarding electroporation and iontophoresis, see Zhang et al., “Advances in transdermal insulin delivery,” Adv. Drug. Deliv. Rev. 139:51-70 (2019), the entire contents of which are incorporated by reference herein. In some examples, control electronics 120 apply the electrical stimulus to the first electrode 141 and the second electrode 142 to deliver the therapeutic agent into the epidermis 20 via magnetohydrodynamics. For further details regarding magnetohydrodynamics, see the following references, the entire contents of each of which are incorporated by reference herein: Hakala et al., “Sampling of fluid through skin with magnetohydrodynamics for noninvasive glucose monitoring,” Scientific Reports 11:7609, 9 pages (2021); Park et al., “Soft, smart contact lenses with integrations of wireless circuits, glucose sensors, and displays,” Sci. Adv. 4: eeap9841 (2018); Lemoff et al., “AC magnetohydrodynamic micropump,” Sensors Actuators B Chem 63:178-185 (2000); Jang et al., “Theoretical and experimental study of MHD (magnetohydrodynamic) micropump,” Sens. Actuators A Phys 80:84-89 (2000); Das et al., “Some practical applications of magnetohydrodynamic pumping,” Sens. Actuators A Phys 201:43-48 (2013); and Chang et al., “A needle-free technique for interstitial fluid sample acquisition using a Lorentz-force actuated jet injector,” J. Control. Release 211:37-43 (2015).

In one example, the analyte which is measured by wearable device 100 is glucose, and the therapeutic agent delivered using wearable device 100 is insulin. Insulin typically includes phenolic preservatives, namely, phenol and/or m-cresol, which are electroactive species that can interfere with the signal detected by the sensor. By delivering the insulin to the dermis 30 via iontophoresis, and by sensing the glucose concentration in the subcutaneous tissue 40, interference from the phenolic preservatives is avoided.

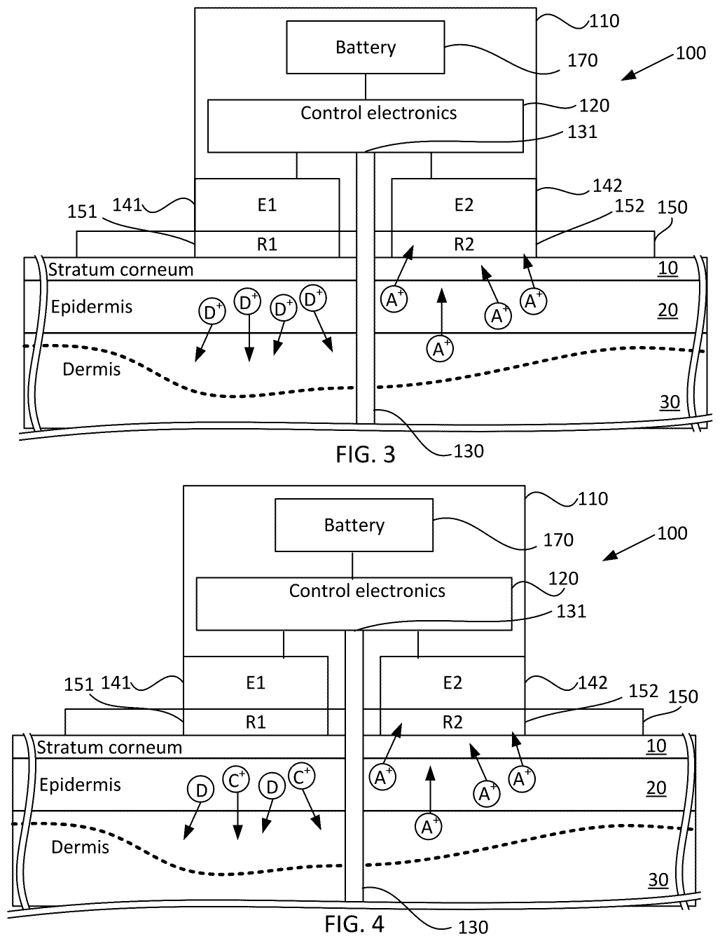

As noted further above, the at least one reservoir of wearable device 100 optionally include a polymer complexed with a therapeutic agent. For example, in the nonlimiting configuration illustrated in FIG. 1A, at least one of the first reservoir 151 and the second reservoir 152 (which also may be denoted R1 and R2, respectively) include a polymer complexed with the therapeutic agent. Such a polymer(s) find particular utility in examples in which the therapeutic agent is delivered via electroporation or by iontophoresis. FIG. 3 schematically illustrates an alternative example configuration of, and operation performed by, a wearable device for delivering a therapeutic agent 160. In the nonlimiting example 101 illustrated in FIG. 3, in which the therapeutic agent 160 is delivered using iontophoresis, a first reservoir 151 includes a first polymer complexed with the therapeutic agent, and second reservoir 152 includes a second polymer which does not store the therapeutic agent. In one example, the therapeutic agent 160 is positively charged, as intended to be represented by “D⁺” in FIG. 3. For example, the molecules of the therapeutic agent 160 themselves are charged, or the therapeutic agent is disposed within a positively charged encapsulant. In one example, the electrical stimulus includes applying a positive charge to a first electrode 141 (which acts as an anode) and applying a negative charge to second electrode 142 (which acts as cathode). The positive charge applied to first electrode 141 repels the positively charged therapeutic agent, and the negative charge applied to second electrode 142 attracts positively charged biological counterions 161. Responsive to the electrical stimulus, the therapeutic agent 160 is transported out of the first reservoir 151, across stratum corneum 10, into epidermis 20, and into dermis 30 in a manner such as described with FIGS. 1B-1E. Additionally, as illustrated in FIG. 3, responsive to the electrical stimulus, a biological counterion 161 is transported out of the epidermis 20 and/or dermis 30, across stratum corneum 10, and into the polymer of second reservoir 152. In one example, the biological counterion 161 is positively charged, as intended to be represented by “A⁺” in FIG. 3. In some examples, the electrical stimulus is substantially constant (direct current, DC) to transport the therapeutic agent 160 out of the first reservoir 151 and transport the counterion 161 into the second reservoir 152. In other examples, the electrical stimulus alternates (alternating current, AC) as a function of time to alternately transport the therapeutic agent 160 out of the first reservoir 151 and transport a counterion 161 into the second reservoir 152.

FIG. 4 schematically illustrates another alternative example configuration of, and operation performed by, a wearable device 100 for delivering a therapeutic agent 160. In the nonlimiting example 102 illustrated in FIG. 4, in which the therapeutic agent also is delivered via iontophoresis by first reservoir 151 that includes a first polymer complexed with the therapeutic agent 160, and second reservoir 152 includes a second polymer which does not store the therapeutic agent. In this example, the therapeutic agent 160 has no charge (i.e., it is neutral), as intended to be represented by “D” in FIG. 4, and, in a further example, is mixed with a positively charged carrier 162, intended to be represented by “C⁺” in FIG. 4. The positive charge applied to the first electrode 141 repels the positively charged therapeutic agent 160, and the negative charge applied to second electrode 142 attracts positively charged biological counterions 161. Responsive to the electrical stimulus, the therapeutic agent 160 is transported out of the first reservoir 151 by being carried by the charged carrier 162, across stratum corneum 10, into epidermis 20, and into dermis 30 in a manner such as described with FIGS. 1B-1E. Additionally, as illustrated in FIG. 4, responsive to the electrical stimulus, a biological counterion 161 is transported out of the epidermis 20 and/or dermis 30, across stratum corneum 10, and into the polymer of the second reservoir 152. In one example, the biological counterion 161 is positively charged, as intended to be represented by “A⁺” in FIG. 4.

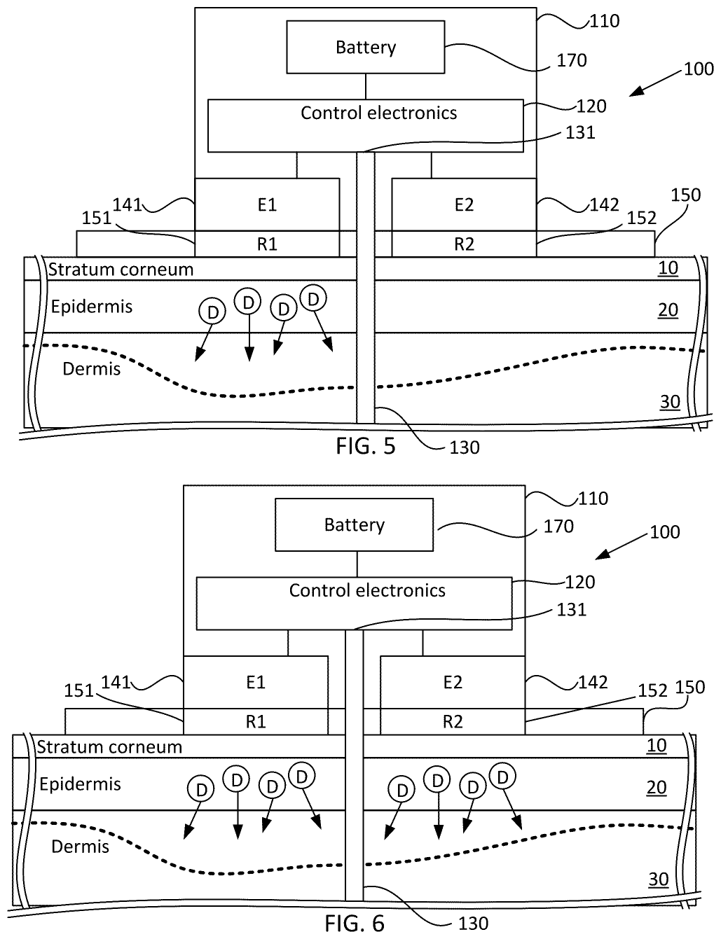

FIG. 5 schematically illustrates an alternative example configuration of, and operation performed by, a wearable device 100 for delivering a therapeutic agent 160. In the nonlimiting example 103 illustrated in FIG. 5, in which the therapeutic agent 160 is delivered using electroporation, a first reservoir 151 includes a first polymer complexed with the therapeutic agent, and a second reservoir 152 includes a second polymer which does not store the therapeutic agent 160. In one example, the therapeutic agent 160 has no charge (i.e., it is neutral), as intended to be represented by “D” in FIG. 5. Responsive to the electrical stimulus, the therapeutic agent 160 is transported out of the first reservoir 151, across stratum corneum 10, into epidermis 20, and into dermis 30 in a manner such as described with FIGS. 1B-1E. In some examples, the electrical stimulus alternates polarity (i.e., alternating current) as a function of time to alternately transport the therapeutic agent out of the first reservoir 151. A first polymer and a second polymer can be polymers such as hydrogels that provide for improved ohmic conductivity (reduced resistance) between the electrode/reservoir and the stratum corneum 10. For example, the polymers provide enhanced impedance matching, absent which a majority of the voltage drop likely occurs between the electrode and the stratum corneum, which can lead to erythema, and potentially a burn.

FIG. 6 schematically illustrates an alternative example configuration of, and operation performed by, a wearable device 100 for delivering a therapeutic agent 160. In the nonlimiting example 104 illustrated in FIG. 6, in which the therapeutic agent 160 is delivered using electroporation, a first reservoir 151 includes a first polymer complexed with the therapeutic agent 160, and a second reservoir 152 includes the same polymer also storing the therapeutic agent 160. In one example, the therapeutic agent 160 has no charge (i.e., it is neutral), as intended to be represented by “D” in FIG. 6. Responsive to the electrical stimulus, the therapeutic agent 160 is transported out of reservoir 151 and out of reservoir 152, across stratum corneum 10, into epidermis 20, and into dermis 30 in a manner such as described with FIGS. 1B-1E. In some examples, the electrical stimulus alternates polarity (i.e., alternating current) as a function of time to alternately transport the therapeutic agent out of the first reservoir 151 and the second reservoir 152.

Regardless of the particular form of electrical stimulus and the particular mode via which the therapeutic agent 160 is delivered to the dermis 30, the control electronics 120, in at least one example, is suitably configured to determine the electrical stimulus based on the measured concentration of the analyte within the subcutaneous tissue 40. Illustratively, for example, the control electronics 120 is configured to determine the electrical stimulus responsive to the signal differing from a predetermined value by more a predetermined amount. For example, control electronics 120 include a memory storing a predetermined value corresponding to a “normal” or “target” value of the analyte, and is configured to compare the signal to the predetermined value (e.g., by calculating the difference between the signal and the predetermined value). In one example, the control electronics 120 are configured to determine a time and/or magnitude of the electrical stimulus based on such comparison. For example, the control electronics increase the time to apply the electrical stimulus proportionally with the magnitude of the difference between the signal and the predetermined value. Or, for example, the control electronics increase the magnitude of the electrical stimulus proportionally with the magnitude of the difference between the signal and the predetermined value.

Additionally, regardless of the particular form of electrical stimulus and the particular mode via which the therapeutic agent 160 is delivered to the dermis 30, the control electronics 120, in one example, is suitably configured to determine the electrical stimulus based not only on the measured concentration of the analyte within the subcutaneous tissue 40, but based on one or more other factors as well. For example, control electronics 120 is configured to determine the electrical stimulus based on a duration of time for which the at least one reservoir (i.e., first reservoir 151 and/or second reservoir 152) has been coupled to the stratum corneum 10. Illustratively, the concentration of the therapeutic agent 160 within the at least one reservoir decreases over time, as the therapeutic agent 160 is delivered to the host. In one example, the control electronics 120 are configured to adjust the electrical stimulus so as to compensate for such depletion of the therapeutic agent 160, so as to provide consistent and accurate dosing of the therapeutic agent 160. For example, control electronics 120 are configured to increase a duration of the electrical stimulus as the duration of time for which the at least one reservoir has been coupled to the epidermis increases. Additionally, or alternatively, control electronics 120 are configured to increase a magnitude of the electrical stimulus as the duration of time for which the at least one reservoir has been coupled to the epidermis increases.

Note that in examples such as described with reference to FIGS. 1A-1E, 2A-2B, and 3-6, the electrical stimulus and dosing of the therapeutic agent 160 substantially does not interfere with the signal corresponding to the concentration of the analyte within the subcutaneous tissue 40. For example, referring again to FIG. 1B, electrical field lines 153 are generated at a location which is sufficiently spaced from distal end 132 of sensor 130 that the field line strength is negligible at the distal end and substantially does not affect the measurement that is made using the distal end 132. Additionally, or alternatively, in some examples control electronics 120 receives the signal from distal end 132 at a time during which the electrical stimulus is not being applied. As such, at the time at which the signal is generated, electrical field lines 153 do not exist and therefore do not interfere with the signal. In some examples, the proximal end 131 of the sensor 130 is less than about 1 cm away from at least one of the first electrode 141 and the second electrode 142.

Although FIGS. 1A-1E, 2A-2B, and 3-6 illustrate a non-limiting example in which the sensor is located between the first electrode 141 and the second electrode 142, it will be appreciated that a portion of the sensor 130 and the first electrode 141 and the second electrode 142 can have any suitable arrangement relative to one another. For example, FIGS. 7A-7B, 8A-8B, and 9A-9B schematically illustrate additional alternative example configurations of, and operations performed by, a wearable device 100 for delivering a therapeutic agent 160.

FIGS. 7A, depicting a side view of wearable device 102 and FIG. 7B depicting a bottom view of the device of FIG. 7A, illustrate an example in which the second electrode 142 (E2) is located between a portion of the sensor 130 and the first electrode 141 (E1). In one example, the wearable device 102 comprises a footplate 150, and housing 110 centrally positioned on footplate 150. In one example, footplate 150 comprises a medical grade adhesive surface configured to secure to the epidermis. In one example, footplate 150 comprises one or more adhesive pads 32 having adhesive surfaces applied to the bottom of the footplate 150 such that, during operation, the pads 32 adhere to the skin of the patient when the wearable device 100 is in use, thereby assisting in securing the housing 110 to the patient's skin so as to prevent the wearable device 100 from shifting position on the skin when in use. The size and shape of the pads 32 may be determined relative to the size and/or weight of wearable device 102 and/or the part of the body to which the wearable device 102 is being applied. The size of the pads may also be determined relative to the type of adhesive being used on the pads. In one example, the one or more adhesive pads 32 are of a rectangular or band shape projecting away from opposite sides of housing 110. In one example, the one or more adhesive pads 32 include peel-away release layers for protecting the adhesive until the wearable device 100 is deployed.

In one example, the releasing force from the patient's skin by the one or more pads 32 is greater than the compressive force applied on a puncture site by the sensor 130. Typical temporary medical adhesives may be used such that when the lifetime of the sensor 130 is achieved, the wearable device 100 is easily removed. Optionally, the at least one reservoir (e.g., reservoirs 151 and 152) is located within or surrounded by the adhesive pad 32 as shown in FIG. 7B.

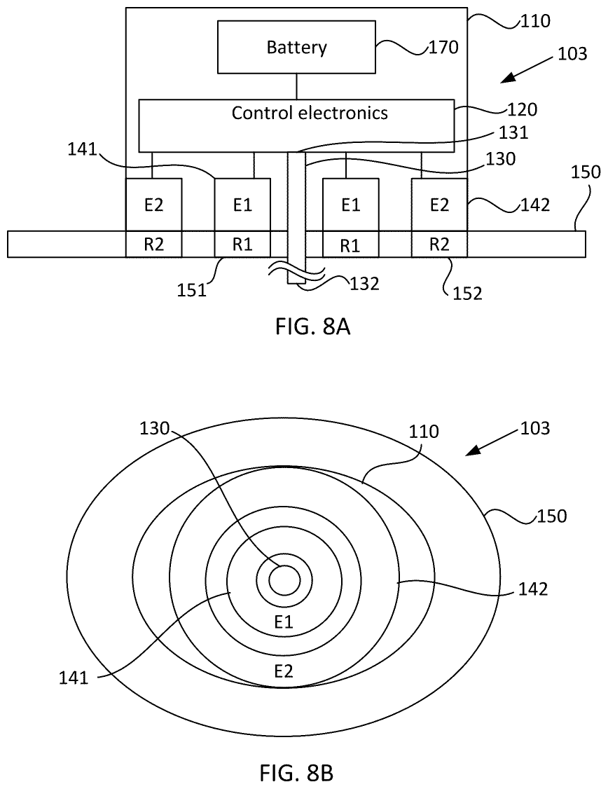

FIGS. 8A, depicting a side view of wearable device 103 and FIG. 8B depicting a bottom view of the device of FIG. 8A illustrate an example in which the sensor 130 is located in an aperture within the first electrode 141 (E1). Optionally, as illustrated in FIGS. 8A-8B, the first electrode 141 is located in an aperture within the second electrode 142 (E2).

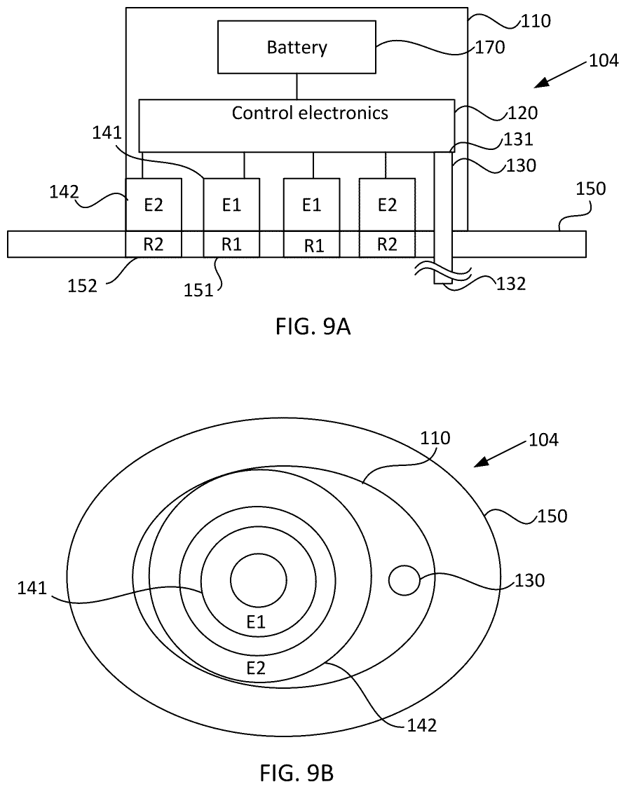

FIGS. 9A, depicting a side view of wearable device 104 and FIG. 9B depicting a bottom view of the device of FIG. 9A illustrate another example in which the second electrode 142 (E2) is located between a portion of the sensor 130 and the first electrode 141 (E1). In this example, a portion of the sensor 130 is located outside of both the first electrode 141 and the second electrode 142, and the first electrode 141 is located in an aperture within the second electrode 142.

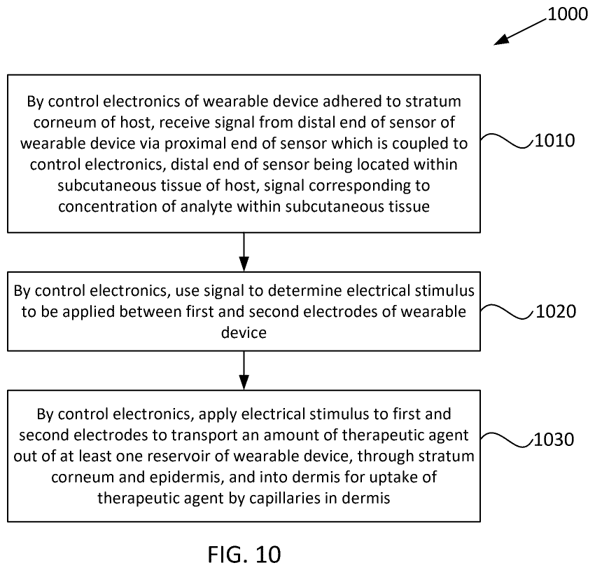

As should be appreciated from the present disclosure, the present wearable device can have any of a variety of suitable configurations, and can be used to implement any suitable operations. For example, FIG. 10 illustrates a flow of operations in an example method for delivering a therapeutic agent using a wearable device. Method 1000 illustrated in FIG. 10 includes receiving a signal by control electronics of a wearable device coupled to stratum corneum of a host from a distal end of a sensor via a proximal end of the sensor that is coupled to the control electronics, the distal end of the sensor being located within subcutaneous tissue of the host, the signal corresponding to the concentration of the analyte within the subcutaneous tissue (operation 1010). Nonlimiting examples of such a device, sensor, and adhesive are described with reference to FIGS. 1A-1E, 2A-2B, 3-6, and 7A-9B. Method 1000 illustrated in FIG. 10 includes determining an electrical stimulus using the signal received by the control electronics that is to be applied between a first electrode and a second electrode of the wearable device (operation 1020). Nonlimiting examples of the manner in which the control electronics are use the signal to determine the electrical stimulus are provided elsewhere herein. Method 1000 illustrated in FIG. 10 includes applying the electrical stimulus determined by the control electronics to the first electrode and the second electrode (operation 1030). Method 1000 illustrated in FIG. 10 includes transporting an amount of the therapeutic agent out of at least one reservoir of the wearable device, through the stratum corneum, and into the dermis for uptake of the therapeutic agent by capillaries in the dermis (operation 1040). Nonlimiting examples of electrical stimuli, and the manner in which such electrical stimuli is used to transport a therapeutic agent from a reservoir through the epidermis and into the dermis, are described elsewhere herein.

Sensor 130 optionally is configured in such a manner as to enhance its biocompatibility. For example, the biocompatibility of sensor 130 optionally is enhanced by providing a biointerface membrane (not specifically illustrated) over one or more component(s) of sensor 130. In some examples, the biointerface membrane is configured to inhibit biofouling of sensor 130. Nonlimiting examples of materials which are included in the biointerface membrane(s) include hard segments and/or soft segments. Examples of hard and soft segments used for the biointerface membrane include aromatic polyurethane hard segments with Si groups, aliphatic hard segments, polycarbonate soft segments or any combination thereof. In some examples of biointerface membrane(s), polyvinylpyrrolidone (PVP) are not be included. In this example where no PVP is included, the biointerface membrane includes polyurethane and poly(dimethylsiloxane) (PDMS). In some examples, which are combined with other examples herein, the biointerface membranes discussed herein includes one or more zwitterionic compounds.

Additionally, or alternatively, the biointerface membrane(s) is/are configured to release a therapeutic compound into the biological fluid. In one example, the therapeutic compounds suitable for release using the biointerface membrane(s) or other membranes as discussed herein includes one or more of anti-inflammatory agents, anti-infective agents, necrosing agents, and anesthetics. Generally, anti-inflammatory agents reduce acute and/or chronic inflammation adjacent to the implant, in order to decrease the formation of a FBC capsule to reduce or prevent barrier cell layer formation. Suitable anti-inflammatory agents include but are not limited to, for example, nonsteroidal anti-inflammatory drugs (NSAIDs) such as acetaminophen, aminosalicylic acid, aspirin, celecoxib, choline magnesium trisalicylate, diclofenac potassium, diclofenac sodium, diflunisal, etodolac, fenoprofen, flurbiprofen, ibuprofen, indomethacin, interleukin (IL)-10, IL-6 mutein, anti-IL-6 iNOS inhibitors (for example, L-NAME or L-NMDA), interferon, ketoprofen, ketorolac, leflunomide, melenamic acid, mycophenolic acid, mizoribine, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, paclitaxel, tacrolimus, tranilast, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, dexamethasone, and dexamethasone acetate.

Generally, immunosuppressive and/or immunomodulatory agents interfere directly with several key mechanisms necessary for involvement of different cellular elements in the inflammatory response. Suitable immunosuppressive and/or immunomodulatory agents include anti-proliferative, cell-cycle inhibitors, (for example, paclitaxol (e.g., Sirolimus), cytochalasin D, infiximab), taxol, actinomycin, mitomycin, thospromote VEGF, estradiols, NO donors, QP-2, tacrolimus, tranilast, actinomycin, everolimus, methothrexate, mycophenolic acid, angiopeptin, vincristing, mitomycine, statins, C MYC antisense, sirolimus (and analogs), RestenASE, 2-chloro-deoxyadenosine, PCNA Ribozyme, batimstat, prolyl hydroxylase inhibitors, PPARγ ligands (for example troglitazone, rosiglitazone, pioglitazone), halofuginone, C-proteinase inhibitors, probucol, BCP671, EPC antibodies, catchins, glycating agents, endothelin inhibitors (for example, Ambrisentan, Tesosentan, Bosentan), Statins (for example, Cerivasttin), E. coli heat-labile enterotoxin, NLRP3 inflammasome inhibitors, and advanced coatings.

Generally, anti-infective agents are substances capable of acting against infection by inhibiting the spread of an infectious agent or by killing the infectious agent outright, which can serve to reduce immuno-response without inflammatory response at the implant site. Anti-infective agents include, but are not limited to, anthelmintics (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin; clindamycin; colistimethate sodium; polymyxin b sulfate; vancomycin; antivirals including acyclovir, amantadine, didanosine, efavirenz, foscarnet, ganciclovir, indinavir, lamivudine, nelfinavir, ritonavir, saquinavir, silver, stavudine, valacyclovir, valganciclovir, zidovudine; quinolones (ciprofloxacin, levofloxacin); sulfonamides (sulfadiazine, sulfisoxazole); sulfones (dapsone); furazolidone; metronidazole; pentamidine; sulfanilamidum crystallinum; gatifloxacin; and sulfamethoxazole/trimethoprim.

Generally, necrosing agents are any drug that causes tissue necrosis or cell death. Necrosing agents include cisplatin, BCNU, taxol or taxol derivatives, and the like.

Generally, vascularization agents include substances with direct or indirect angiogenic properties. In some cases, vascularization agents additionally affect formation of barrier cells in vivo. By indirect angiogenesis, it is meant that the angiogenesis can be mediated through inflammatory or immune stimulatory pathways. It is not fully known how agents that induce local vascularization indirectly inhibit barrier-cell formation; however it is believed that some barrier-cell effects can result indirectly from the effects of vascularization agents.

Vascularization agents include mechanisms that promote neovascularization around the membrane and/or reduce or minimize periods of ischemia by increasing vascularization close to the device-tissue interface. Sphingosine-1-Phosphate (S1P), which is a phospholipid possessing potent angiogenic activity, is incorporated into a biointerface membrane of a nonlimiting example. Monobutyrin, which is a potent vasodilator and angiogenic lipid product of adipocytes, is incorporated into a biointerface membrane of another nonlimiting example. In another nonlimiting example, an anti-sense molecule (for example, thrombospondin-2 anti-sense), which increases vascularization, is incorporated into a biointerface membrane.

Vascularization agents can include mechanisms that promote inflammation, which is believed to cause accelerated neovascularization in vivo. In one nonlimiting example, a xenogenic carrier, for example, bovine collagen, which by its foreign nature invokes an immune response, stimulates neovascularization, and is incorporated into a biointerface membrane of the present disclosure. In another nonlimiting example, Lipopolysaccharide, which is a potent immunostimulant, is incorporated into a biointerface membrane. In another nonlimiting example, a protein, for example, a bone morphogenetic protein (BMP), which is known to modulate bone healing in tissue, is incorporated into a biointerface membrane.

Generally, angiogenic agents are substances capable of stimulating neovascularization, which can accelerate and sustain the development of a vascularized tissue bed at the device-tissue interface. Angiogenic agents include, but are not limited to, copper ions, iron ions, tridodecylmethylammonium chloride, Basic Fibroblast Growth Factor (bFGF), (also known as Heparin Binding Growth Factor-II and Fibroblast Growth Factor II), Acidic Fibroblast Growth Factor (aFGF), (also known as Heparin Binding Growth Factor-I and Fibroblast Growth Factor-I), Vascular Endothelial Growth Factor (VEGF), Platelet Derived Endothelial Cell Growth Factor BB (PDEGF-BB), Angiopoietin-1, Transforming Growth Factor Beta (TGF-Beta), Transforming Growth Factor Alpha (TGF-Alpha), Hepatocyte Growth Factor, Tumor Necrosis Factor-Alpha (TNF-Alpha), Placental Growth Factor (PLGF), Angiogenin, Interleukin-8 (IL-8), Hypoxia Inducible Factor-I (HIF-1), Angiotensin-Converting Enzyme (ACE) Inhibitor Quinaprilat, Angiotropin, Thrombospondin, Peptide KGHK, Low Oxygen Tension, Lactic Acid, Insulin, Copper Sulphate, Estradiol, prostaglandins, cox inhibitors, endothelial cell binding agents (for example, decorin or vimentin), glenipin, hydrogen peroxide, nicotine, and Growth Hormone.

Generally, pro-inflammatory agents are substances capable of stimulating an immune response in host tissue, which can accelerate or sustain formation of a mature vascularized tissue bed. For example, pro-inflammatory agents are generally irritants or other substances that induce chronic inflammation and chronic granular response at the implantation-site. While not wishing to be bound by theory, it is believed that formation of high tissue granulation induces blood vessels, which supply an adequate or rich supply of analytes to the device-tissue interface. Pro-inflammatory agents include, but are not limited to, xenogenic carriers, Lipopolysaccharides, S. aureus peptidoglycan, and proteins.

Other substances that can be incorporated into one or more membranes of the present disclosure include various pharmacological agents, excipients, and other substances well known in the art of pharmaceutical formulations.

Additionally, or alternatively, in some examples, the biointerface membrane(s) include a biocompatible polymer. In some examples, the biocompatible polymer is selected from the group consisting of: polyvinyl butyral (PVB) or polyurethane. In certain examples, the biocompatible polymer can be a segmented block copolymer. In one example, the segmented block copolymer includes hard segments and soft segments. In this example, the hard segments include aromatic or aliphatic diisocyanates are used to prepare hard segments of segmented block copolymer. In one example, the aliphatic or aromatic diisocyanate used to provide hard segment of polymer includes one or more of norbornane diisocyanate (NBDI), isophorone diisocyanate (IPDI), tolylene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-diphenylmethane diisocyanate (MDI), trans-1,4-bis(isocyanatomethyl) cyclohexane (1,4-H6XDI), 1,4-cyclohexyl diisocyanate (CHDI), 1,4-phenylene diisocyanate (PPDI), 3,3′-dimethyl-4,4′-biphenyldiisocyanate (TODI), 1,6-hexamethylene diisocyanate (HDI), or combinations thereof.

In one example, the hard segment content is from about 5 wt. % to about 90 wt. % of the segmented block copolymer of the biointerface membrane. In another example, the hard segments is from about 15 wt. % to about 75 wt. %. In yet another example, the hard segments is from about 25 wt. % to about 55 wt. %.

It will be appreciated that the biointerface membrane(s) can include a plurality of layers.

It will further be appreciated that sensor 130 can have any suitable configuration. In the nonlimiting example illustrated in FIG. 1A, sensor 130 is substantially coaxially shaped and is referred to as a “wire.” Sensor 130 can alternatively be substantially planar in shape and is referred to as a “flat sensor.”

Further details regarding example configurations of control electronics 120 now will be provided. In one example, the control electronics 120 include circuitry such as a non-volatile computer-readable memory configured to store, among other measures, a time series data set of raw signal values in volts, amperes, or ohms (which correspond to measured analyte concentrations) and electrical stimuli to be applied that delivers a dose of the therapeutic agent which is appropriate based on the measured analyte concentration. Note, however, that the control electronics 120 need not be configured to determine the actual concentration of the analyte within the subcutaneous tissue, and similarly need not be configured to determine the actual dose of the therapeutic agent to be delivered. Rather, in one example, control electronics 120 are configured to apply an electrical stimulus based on the signal having a particular value.

Optionally, in addition to applying the electrical stimulus, control electronics 120 are configured to generate an output, based on the signal, corresponding to the concentration of the analyte within the subcutaneous tissue. Such output can be used in any suitable manner. In some examples, control electronics 120 include a non-volatile computer-readable memory configured to store the output or a microprocessor or digital signal processor configured to run a signal processing algorithm. Additionally, or alternatively, in some examples, control electronics 120 includes a transmitter configured to wirelessly transmit an output and receive an input, e.g., a near-field communication (NFC), Bluetooth, WiFi, or cellular transmitter. The output can be used in any suitable manner, e.g., so as to continuously monitor one or more indicators of the host's health. In one example, the control electronics 120 receive an input via a transmitter from an algorithm running on a remote processor (e.g. cloud computing) and translates the input to a signal that delivers the therapeutic agent.

A processor module includes the central control unit that controls the processing of the control electronics. In some examples, the processor module includes a microprocessor, however a computer system other than a microprocessor can be used to process data as described herein, for example an application-specific integrated circuit (ASIC), field-programmable gate array (FPGA), central processing unit (CPU), or graphical processing unit (GPU) can be used for some or all of the sensor's central processing. In one example, the processor is coupled to a computer-readable memory via which the processor is configured to provide semi-permanent storage of data, for example, storing data such as sensor identifier (ID) and programming to process data streams (for example, programming for data smoothing and/or replacement of signal artifacts similar to that described in U.S. Pat. No. 820,174 to Goode et al., incorporated by reference in its entirety herein). The processor additionally can be used for the system's cache memory, for example for temporarily storing recent sensor data. In some examples, the processor module is coupled to one or more computer-readable memory storage components such as ROM, RAM, dynamic-RAM, static-RAM, non-static RAM, EEPROM, rewritable ROMs, OCM, OTP memory, flash memory, or the like.

In some examples, the processor module includes an analog-to-digital (A/D) converter configured to convert an analog signal, received from distal tip or end 132, into a digital signal for analysis. In one example, the processor module also includes a digital filter, for example, an IIR or FIR filter, configured to smooth the raw data stream from the A/D converter. In some examples, digital filters are programmed to filter data sampled at a predetermined time interval (also referred to as a sample rate). In some examples, wherein the control electronics are configured to use the distal end 132 measure the analyte at discrete time intervals, these time intervals determine the sample rate of the digital filter. In some alternative examples, wherein the control electronics are configured to use the distal end 132 continuously measure the analyte, the processor module can be programmed to request a digital value from the A/D converter at a predetermined time interval, also referred to as the acquisition time. In these alternative examples, the values obtained by the processor are advantageously averaged over the acquisition time due the continuity of the measurement. Accordingly, the acquisition time determines the sample rate of the digital filter. In one example, the processor module is configured with a programmable acquisition time, namely, the predetermined time interval for requesting the digital value from the A/D converter is programmable by a user within the digital circuitry of the processor module. An acquisition time of from about 2 seconds to about 512 seconds is used in some examples; however, any acquisition time can be programmed into the processor module. A programmable acquisition time is advantageous in optimizing noise filtration, time lag, and processing/battery power.

In one example, a battery 170 is operably connected to the control electronics and provides the power for the wearable device. In one example, the battery is a lithium manganese dioxide battery; however, any appropriately sized and powered battery can be used (for example, AAA, coin cell, nickel-cadmium, zinc-carbon, alkaline, lithium, nickel-metal hydride, lithium-ion, zinc-air, zinc-mercury oxide, silver-oxide, silver-zinc, and/or hermetically-sealed). In some examples, the battery is rechargeable, and/or a plurality of batteries can be used to power the system. In some examples, the wearable device can be powered via an inductive coupling, for example. In some examples, a quartz crystal and/or real-time clock (RTC) is operably connected to the processor and maintains system time for the computer system as a whole, for example for the programmable acquisition time within the processor module.

In some examples, output signal (from the control electronics) is sent to a receiver (e.g., a computer or other communication station). The output signal may, in some examples, include a raw data stream that is used to provide a useful value of the measured analyte concentration to a patient or a doctor, for example. In some examples, the raw data stream can be continuously or periodically algorithmically smoothed or otherwise modified to diminish outlying points that do not accurately represent the analyte concentration, for example due to signal noise or other signal artifacts, in a manner such as described in U.S. Pat. No. 810,174 to Goode et al., which is incorporated herein by reference in its entirety.

When a sensor is first implanted into host tissue, the sensor and receiver are initialized. This can be referred to as start-up mode, and involves optionally resetting the sensor data and calibrating the sensor. In selected examples, mating the electronics unit to the mounting unit triggers a start-up mode. In other examples, the start-up mode is triggered by the receiver.

In some examples, the control electronics are wirelessly connected to a receiver via one- or two-way RF transmissions or the like. However, a wired connection is also contemplated. The receiver provides much of the processing and display of the sensor data, and can be selectively worn and/or removed at the host's convenience. Thus, the sensor system can be discreetly worn, and the receiver, which provides much of the processing and display of the sensor data, can be selectively worn and/or removed at the host's convenience. Particularly, the receiver includes programming for retrospectively and/or prospectively initiating a calibration, converting sensor data, updating the calibration, evaluating received reference and sensor data, and evaluating the calibration for the analyte sensor, in a manner such as described in U.S. Pat. No. 7,778,680, which is incorporated herein by reference in its entirety.

In some examples, control electronics 120 can be affixed to a printed circuit board (PCB), or the like, and can take a variety of forms. For example, the control electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

In some examples, control electronics 120 include a regulated current source (e.g., in examples in which the therapeutic agent is delivered using iontophoresis), or includes a waveform generator (e.g., in examples in which the therapeutic agent is delivered using electroporation). In one example, the current source is voltage controlled, and compliance limits are established to limit voltage and/or current so as to limit harm to the host. In one example, the waveform generator is a direct digital synthesis variety and also is regulated to limit current and/or voltage. Alternatively, the waveform generator is a simple sine wave implementation, e.g., a voltage-controlled oscillator.

### Additional Comments

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific examples disclosed herein, but that it covers all modifications and alternatives coming within the true scope and spirit of the disclosure.

While certain examples of the present disclosure have been illustrated with reference to specific combinations of elements, various other combinations may also be provided without departing from the teachings of the present disclosure. Thus, the present disclosure should not be construed as being limited to the particular examples described herein and illustrated in the Figures, but may also encompass combinations of elements of the various illustrated examples and aspects thereof.

What is claimed is:

1. A wearable device for sensing a concentration of an analyte and delivering a therapeutic agent, the wearable device comprising:
a sensor configured to extend fully through stratum corneum, epidermis, and dermis of a host and partially into subcutaneous tissue of the host,
the sensor comprising a proximal end and a distal end,
the distal end configured to be located within the subcutaneous tissue;
at least one reservoir configured to contact the stratum corneum and comprising the therapeutic agent; and
control electronics coupled to the proximal end of the sensor and comprising a first electrode and a second electrode, the control electronics configured to:
via the proximal end of the sensor, receive a signal from the distal end of the sensor corresponding to the concentration of the analyte within the subcutaneous tissue,
determine, using the signal, an electrical stimulus to be applied to the first electrode and the second electrode, and
apply the electrical stimulus to the first electrode and the second electrode to deliver the therapeutic agent across the stratum corneum and into the dermis of the host.

2. The wearable device of claim 1, wherein the electrical stimulus:
transports an amount of the therapeutic agent out of a polymer;
increases permeability of the stratum corneum to transport the amount of the therapeutic agent into the dermis;
systemically distributes the amount of the therapeutic agent transported through the stratum corneum into the dermis;
delivers the therapeutic agent through the stratum corneum and into the dermis via iontophoresis;
delivers the therapeutic agent into the dermis via magnetohydrodynamics.

3. The wearable device of claim 1, wherein the therapeutic agent is neutrally charged, positively charged, negatively charged, or a charged carrier contains the therapeutic agent, wherein the charged carrier is positively or negatively charged.

4. The wearable device of claim 1, wherein the at least one reservoir comprises a first reservoir adjacent to the first electrode or wherein the at least one reservoir further comprises a second reservoir adjacent to the second electrode.

5. The wearable device of claim 1, wherein both the first reservoir and the second reservoir comprise a polymer complexed with the therapeutic agent.

6. The wearable device of claim 5, wherein the electrical stimulus:
alternates polarity as a function of time to alternately transport the therapeutic agent out of the first reservoir and the second reservoir;
alternates polarity as a function of time to transport the therapeutic agent out of the first reservoir and to transport a counterion into the second reservoir;
substantially does not interfere with the signal corresponding to the concentration of the analyte within the subcutaneous tissue;

7. The wearable device of claim 1, wherein the control electronics receives the signal from the sensor at a time during which the electrical stimulus is not being applied.

8. The wearable device of claim 1, wherein the control electronics is configured to:
determine a parameter of the electrical stimulus based on a duration of time for which the at least one reservoir has been coupled to the stratum corneum;
increase a duration of applying the electrical stimulus as the duration of time for which the at least one reservoir has been coupled to the stratum corneum increases;
increase a magnitude of the electrical stimulus as the duration of time for which the at least one reservoir has been coupled to the stratum corneum increases;
determine the electrical stimulus responsive to the signal corresponding to the concentration of the analyte differing from a predetermined value by more a predetermined amount.

9. The wearable device of claim 1, wherein the analyte comprises at least one of insulin, levodopa, metformin, glucagon, GLP-1 antagonist, SGLT-2 inhibitor, vancomycin, gentamycin, epinephrine, or naloxone, or a metabolite thereof.

10. The wearable device of claim 1, wherein an adhesive is configured to adhere the wearable device to the stratum corneum, wherein the at least one reservoir is located within the adhesive.

11. A method for sensing a concentration of an analyte and delivering a therapeutic agent, the method comprising:
receiving a signal by control electronics of a wearable device coupled to stratum corneum of a host from a distal end of a sensor via a proximal end of the sensor that is coupled to the control electronics, the distal end of the sensor being located within subcutaneous tissue of the host, the signal corresponding to the concentration of the analyte within the subcutaneous tissue;
determining an electrical stimulus using the signal received by the control electronics that is to be applied between a first electrode and a second electrode of the wearable device;
applying the electrical stimulus determined by the control electronics to the first electrode and the second electrode; and
transporting an amount of the therapeutic agent out of at least one reservoir of the wearable device, through the stratum corneum, and into the dermis for uptake of the therapeutic agent by capillaries in the dermis.

12. The method of claim 11, wherein transporting an amount of the therapeutic agent out of the at least one reservoir of the wearable device comprises transporting the therapeutic agent out of a polymer.

13. The method of claim 11, wherein applying the electrical stimulus determined by the control electronics to the first electrode and the second electrode comprises increasing permeability of the stratum corneum.

14. The method of claim 11, wherein transporting the amount of the therapeutic agent via iontophoresis, electroporation or magnetohydrodynamics.

15. The method of claim 11, wherein applying the electrical stimulus determined by the control electronics to the first electrode and the second electrode comprises alternating polarity as a function of time to alternately transport the therapeutic agent out of a first reservoir or a second reservoir.

16. The method of claim 11, wherein applying the electrical stimulus comprises alternating polarity as a function of time, or at a substantially constant polarity, so as to transport the therapeutic agent out of the at least one reservoir.

17. The method of claim 11, wherein the electrical stimulus substantially does not interfere with the signal corresponding to the concentration of the analyte within the subcutaneous tissue.

18. The method of claim 11, wherein receiving a signal by control electronics and applying the electrical stimulus determined by the control electronics occur at different times.

19. The method of claim 11, further comprising determining a parameter of the electrical stimulus by control electronics based on a duration of time for which the at least one reservoir has been coupled to the stratum corneum.

20. The method of claim 11, wherein the determining an electrical stimulus using the signal received by the control electronics that is to be applied between a first electrode and a second electrode of the wearable device comprises comparing the concentration of the analyte to a predetermined value.
